(19) United States
(12) Patent Application Publication
(10) Pub. No.: US 2010/0152859 A1
(43) Pub. Date: Jun. 17, 2010

(54) Femoral Implant For Hip Arthroplasty

(76) Inventors: Matthew T. Thompson, Houston, TX (US); Rikin V. Patel, Cypress, TX (US)

Correspondence Address: CONLEY ROSE, P.C.;David A. Rose, P. O. BOX 3267, HOUSTON, TX, 77253-3267, US

(21) Appl. No.: 12/608,797

(22) Filed: Oct. 29, 2009

Related U.S. Application Data
(60) Provisional application No. 61/109,227, filed on Oct. 29, 2008.

(51) Int. Cl. A61F 2/32

(52) U.S. Cl. 623/20.36; 623/20.35

(57) ABSTRACT

A prosthetic femoral implant for use in hip arthroplasty comprises an elongate femoral stem. In addition, the femoral implant comprises a femoral neck having a central axis, a first end integral with the femoral stem, and a second end distal the femoral stem. A transverse cross-section of the femoral neck includes a medial-lateral axis and an anterior-posterior axis. Moreover, a reference circle bisected by the medial-lateral axis and passing through the medial-most point and the lateral-most point has a diameter equal to a maximum medial-lateral width Wₘₗ of the transverse cross-section and an area A₁. The lateral-most anterior segment of the transverse cross-section includes a laterally expanded area extending outside the reference circle, the laterally expanded area having an area A₂ that is at least 7% of one-fourth of the area A₁ of the reference circle.

### CROSS-REFERENCE TO RELATED APPLICATIONS

This application claims benefit of U.S. provisional application Ser. No. 61/109,227 filed Oct. 29, 2008, and entitled “Femoral Implant with Improved Range of Joint Motion,” which is hereby incorporated herein by reference in its entirety for all purposes.

### STATEMENT REGARDING FEDERALLY SPONSORED RESEARCH OR DEVELOPMENT

Not applicable.

### BACKGROUND

1. Field of the Invention

The invention relates generally to implants. More particularly, the invention relates to a femoral implant to enhance the range of motion of the hip joint following a total hip arthroplasty.

2. Background of the Invention

Although it is intended that a total hip replacement will fully restore the normal range of motion and ease of movement of the hip joint, this goal is rarely achieved in practice. Many artificial hip prostheses allow the patient sufficient motion to perform basic activities such as walking and sitting. However, most conventional hip prostheses do not permit extreme maneuvers with compound rotations of the hip that are becoming more common and desirable as hip replacement patients become progressively younger and increasingly more active. Such complex motions often require the femur to rotate about the hip joint in a plane that is not parallel or perpendicular to the anterior or front of the body. Common activities that necessitate compound rotations include rising from a low chair and picking up objects from the floor when seated. Other activities, such as crossing of the legs in a seated position or rolling over in bed, necessitate significant internal or external rotation of the femur about its longitudinal axis. In each of these situations, conventional artificial hip joints typically allow significantly less range of motion compared to the normal hip joint.

Referring briefly to FIG. 1, a conventional artificial hip joint 10 is shown extending between the pelvis 12 and femur 13 of a patient. Conventional artificial hip joint 10 includes a hemispherical socket implant 20, also referred to as an acetabular cup, positioned in the acetabulum 14 of the pelvis 12 of a patient, and a femoral implant 30 extending from the femur 13 of the patient. The socket implant 20 rotatably mates with and forms a ball-and-socket joint with spherical head 37 of the femoral implant 30. Attempts by the patient to force the artificial hip joint 10 to perform the certain activities and/or complex motions (e.g., compound rotations) may cause impingement of the components of the hip joint 10 and ultimately dislocation of the joint 10. Dislocation of joint 10 results when the head 37 of the femoral implant 30 levers out of the hemispherical socket implant 20. In most cases, the dislocated femoral head 31 migrates to a position posterior to the pelvis 12 with considerable pain and shortening of the limb. Recurrent dislocation often requires surgery to correct the problem.

Accordingly, there remains a need in the art for a femoral implant capable of providing increased range of motion. Such an implant would be particularly well-received if it permitted complex movement and compound rotations with a reduced likelihood of dislocation.

### BRIEF SUMMARY OF THE DISCLOSURE

These and other needs in the art are addressed in one embodiment by a prosthetic femoral implant for use in hip arthroplasty. In an embodiment, the femoral implant comprises an elongate femoral stem. In addition, the femoral implant comprises a femoral neck having a central axis, a first end integral with the femoral stem, and a second end distal the femoral stem. Further, the femoral implant comprises a spherical femoral head coupled to the second end of the femoral neck. A transverse cross-section of the femoral neck taken perpendicular to the central axis has an outer perimeter including a medial edge, a lateral edge opposite the medial edge, an anterior edge, and a posterior edge opposite the anterior edge. The transverse cross-section of the femoral neck includes a medial-lateral axis bisecting the transverse cross-section into an anterior half and a posterior half. The medial-lateral axis intersects a medial-most point along the medial edge and a lateral-most point along the lateral edge, and wherein the transverse cross-section has a maximum medial-lateral width Wₘₗ measured along the medial-lateral axis between the medial edge and the posterior edge. In addition the transverse cross-section includes an anterior-posterior axis perpendicular to the medial-lateral axis and extending from a posterior-most point along the posterior edge to an anterior-most point along the anterior edge. The transverse cross-section has a maximum anterior-posterior width Wₐₚ measured along the anterior-posterior axis between the posterior edge and the anterior edge. The anterior half of the transverse cross-section includes a lateral-most anterior segment extending from the lateral-most point to a reference line. The reference line is perpendicular to the medial-lateral axis and crosses the medial-lateral axis at a distance Dₗ measured along the medial-lateral axis from the lateral-most point. The distance Dₗ is equal to one-fourth the maximum medial-lateral width Wₘₗ. A reference circle bisected by the medial-lateral axis and passing through the medial-most point and the lateral-most point has a diameter equal to the maximum medial-lateral width Wₘₗ of the transverse cross-section and an area A₁. The lateral-most anterior segment of the transverse cross-section includes a laterally expanded area extending outside the reference circle, the laterally expanded area having an area A₂. The area A₂ of the laterally expanded area is at least 7% of one-fourth of the area A₁ of the reference circle.

These and other needs in the art are addressed in another embodiment by a prosthetic femoral implant for use in hip arthroplasty. In an embodiment, the femoral implant comprises an elongate femoral stem. In addition, the femoral implant comprises a femoral neck having a central axis, a first end integral with the femoral stem, and a second end distal the femoral stem. Further, the femoral implant comprises a spherical femoral head coupled to the second end of the femoral neck. A transverse cross-section of the femoral neck taken perpendicular to the central axis has an outer perimeter including a medial edge, a lateral edge opposite the medial edge, an anterior edge, and a posterior edge opposite the anterior edge. The transverse cross-section of the femoral neck includes a medial-lateral axis bisecting the transverse cross-section into an anterior half and a posterior half, wherein the medial-lateral axis intersects a medial-most point along the medial edge and a lateral-most point along the lateral edge. The transverse cross-section has a maximum medial-lateral width Wₘₗ measured along the medial-lateral axis between the medial edge and the posterior edge. In addition, the transverse cross-section includes an anterior-posterior axis perpendicular to the medial-lateral axis and extending from a posterior-most point along the posterior edge to an anterior-most point along the anterior edge. The transverse cross-section has a maximum anterior-posterior width Wₐₚ measured along the anterior-posterior axis between the posterior edge and the anterior edge. The anterior half of the transverse cross-section includes a lateral-most anterior segment extending from the lateral-most point to a reference line. The reference line is perpendicular to the medial-lateral axis and crosses the medial-lateral axis at a distance Dₗ measured along the medial-lateral axis from the lateral-most point. The distance Dₗ is equal to one-fourth the maximum medial-lateral width Wₘₗ. The lateral-most anterior segment of the transverse cross-section has an area A₁. A reference circle bisected by the medial-lateral axis and passing through the medial-most point and the lateral-most point has a diameter equal to the maximum medial-lateral width Wₘₗ of the transverse cross-section. The reference circle includes a lateral-most half quadrant extending from the lateral-most point along the lateral edge to the reference line, the lateral-most half quadrant of the reference circle having an area A₂. The area A₁ of the lateral-most anterior segment of the transverse cross-section is at least 116% of the area A₂ of the lateral-most half quadrant of the reference circle.

In an embodiment, a femoral hip arthroplasty comprises a symmetric neck portion that is optimized to improve range of motion during the complex maneuvers of the hip. The neck includes a cross-sectional shape consisting of areas of locally reduced thickness in regions known to limit joint motion by prosthetic impingement. Further, the cross-sectional shape of the neck includes enlarged portions in areas where motion is limited by soft-tissue factors, prior to the occurrence of prosthetic impingement.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a detailed description of the preferred embodiments of the invention, reference will now be made to the accompanying drawings in which:

FIG. 1 is a partial perspective view of the bones of the human hip and the components of a conventional artificial hip joint;

FIG. 2A is a partial cross-sectional front view of the conventional femoral implant of FIG. 1;

FIG. 2B is an enlarged transverse cross-sectional view of the neck of the femoral implant of FIG. 2A taken along line A-A;

FIGS. 3A-3C are transverse cross-sectional views of the femoral necks of exemplary conventional femoral implants;

FIG. 4A is an enlarged view of the partial cross-section of FIG. 2A illustrating the American Standards of Testing and Measurement standard load testing according to Standard Practice for Cyclic Fatigue Testing of Metallic Stemmed Hip Arthroplasty Femoral Components with Torsion (ASTM F1612-95);

FIG. 4B is an enlarged view of the transverse cross-section of FIG. 2B illustrating the American Standards of Testing and Measurement standard load testing according to Standard Practice for Cyclic Fatigue Testing of Metallic Stemmed Hip Arthroplasty Femoral Components with Torsion (ASTM F1612-95);

FIG. 5 is a transverse cross-sectional view of an ovoid femoral neck of a more recent femoral implant;

FIG. 6A is a partial cross-sectional front view of an embodiment of a femoral implant in accordance with the principles described herein;

FIG. 6B is an enlarged transverse cross-sectional view of the femoral neck of FIG. 6A;

FIGS. 7 and 8 compare the transverse cross-sections of the laterally expanded area of embodiments described herein with the transverse cross-sections of four conventional femoral necks;

FIGS. 9A-9D are transverse cross-sectional views of a conventional conical femoral neck illustrating locations of impingement following the four cases of component orientation described in Example 1;

FIG. 10 is a graphical illustration comparing the range of motion data (in degrees to impingement) of an embodiment of a femoral neck made in accordance with the principles described herein to a conventional 12 mm diameter conical femoral neck of similar strength as described in Example 2; and

FIG. 11 is a graphical illustration of a finite element analysis of maximum principal and von Mises stresses for an embodiment of a femoral neck in accordance with the principles described herein as compared to a conventional 12 mm conical femoral neck as described in Example 3.

### DETAILED DESCRIPTION OF SOME OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices and connections. In addition, as used herein, the terms “axial” and “axially” generally mean along or parallel to a central axis (e.g., central axis of a structure), while the terms “radial” and “radially” generally mean perpendicular to the central axis. For instance, an axial distance refers to a distance measured along or parallel to the central axis, and a radial distance means a distance measured perpendicular to the central axis.

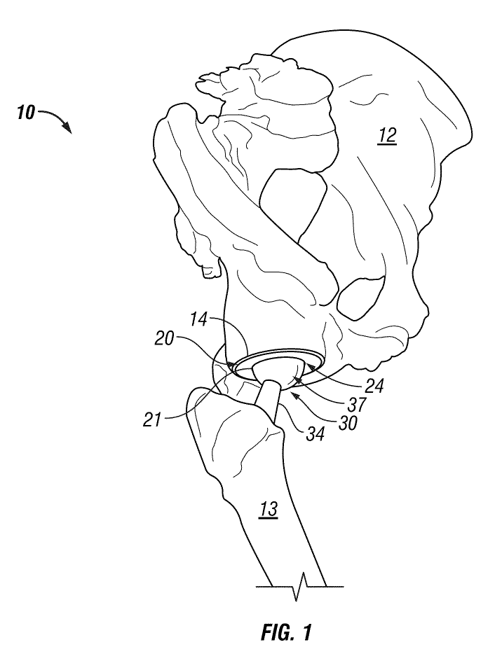

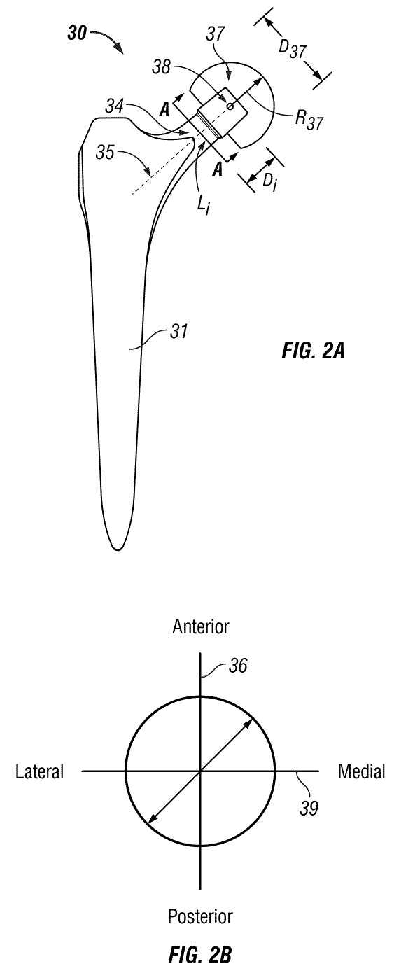

Referring now to FIGS. 1 and 2A, conventional hip joint 10 includes a socket implant 20 and a femoral implant 30. The femoral implant 30 includes an elongate femoral stem 31, a femoral neck 34 integral with and extending from the upper end of the femoral stem 31, and a spherical femoral head 37 fixed to the upper end of the femoral neck 34. The femoral neck 34 has a central or longitudinal axis 35. Further, the spherical femoral head 37 has a geometric center 38 that is equidistant from each point on the spherical surface of the head 37. Further, spherical femoral head 37 has a diameter D₃₇ and a radius R₃₇ equal to one-half the diameter D₃₇. The socket implant or acetabular cup 20 has a spherical receptacle 21 adapted to mate with femoral head 37 to generally form a ball-and-socket joint. The lower portion of the femoral stem 31 is positioned within the upper end of the patient's femur 13, and the acetabular cup 20 is positioned within the patient's hip socket or acetabulum 14. As shown in FIG. 1, the acetabular cup 20 also includes a liner 24 positioned in the receptacle 21 about the femoral head 37. The femoral head 37 is disposed in receptacle 21 of the acetabular cup 20, and slidingly engages the acetabular liner 24 such that the femoral head 37 is free to rotate relative to the acetabular cup 20 within the acetabular liner 24. As shown in FIG. 1, the femur 13 is in a position of extension and external rotation to the point of impinging the pelvis 12, but prior to levering the femoral head 37 out of the acetabular cup 20.

The neck 34 of the femoral implant 30 is the portion inferior to the spherical head 37 that may impinge on the acetabular cup 20 during complex movement and compound rotations. Prosthetic impingement normally occurs at a level Lᵢ disposed at an axial distance Dᵢ measured parallel to axis 35 down the femoral neck 34 from center 38 that is approximately one-half the diameter D₃₇ of the spherical head 37. Depending on the designs of the acetabular cup (e.g., acetabular cup 20) and the femoral head (e.g., femoral head 37), the location of the level (e.g., level Lᵢ) at which impingement may occur typically varies from about 12 mm to 22 mm from the center of the spherical head (e.g., head 37) of the femoral implant (e.g., femoral implant 30) (e.g., distance Di can range from 12 mm to 22 mm). Without being limited by this or any particular theory, the geometry of the transverse cross-section of the femoral neck (e.g., neck 34) at the impingement level (e.g., level Lᵢ) contributes more to the range of motion of the total hip prosthesis (e.g., artificial hip joint 10) than any other feature of the femoral implant (e.g., femoral implant 30). Due to their simplicity and ease of manufacturing, the two most common designs of femoral necks are cylindrical and conical. As used herein, the phrase “transverse cross-section” refers to a cross-section of a structure taken perpendicular to the central or longitudinal axis of the structure. For example, the transverse cross-section of a femoral implant neck is a cross-section taken perpendicular to the central or longitudinal axis of the neck.

Referring now to FIG. 2B, a cross-section taken along line A-A perpendicular to axis 35 at level Lᵢ is shown. The cross-section has an anterior-posterior axis 36, and a medial-lateral axis 39. In general, the medial-lateral axis of a femoral neck is an axis that is orthogonal to the neck axis and extends from the medial-most point of the cross-section of the femoral neck to the lateral-most point of the cross-section of the femoral neck. Further, the anterior-posterior axis of a femoral neck is orthogonal to the neck axis (e.g., neck axis 35) and medial-lateral axis, and extends from the posterior-most point of the cross-section of the femoral neck to the anterior-most point of the cross-section of the femoral neck. As shown in FIG. 2B, both cylindrical and conical conventional femoral neck designs have circular cross-sections taken perpendicular to the neck central axis (e.g., axis 35).

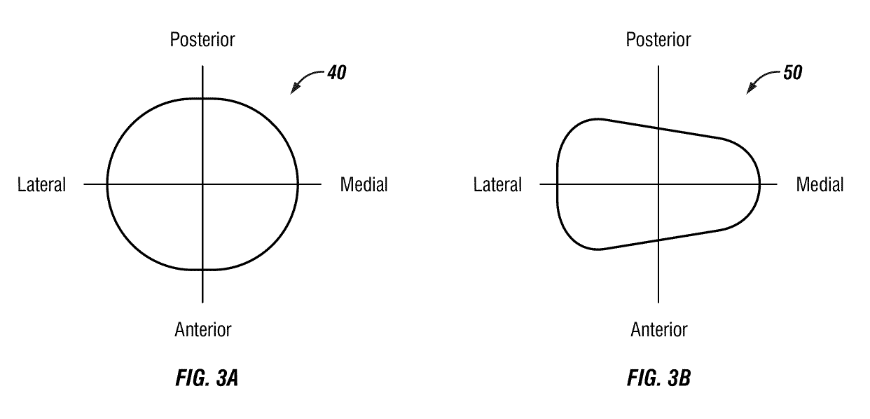

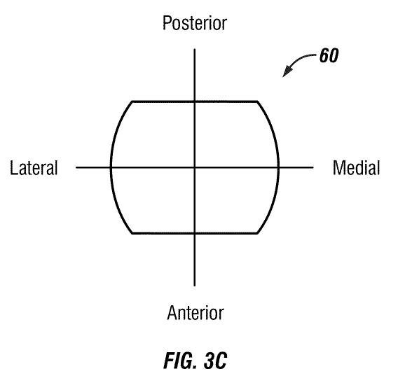

FIGS. 3A-3C illustrate the cross-sections of three other conventional femoral neck designs 40, 50, 60, respectively. The cross-section of each conventional neck design 40, 50, 60 shown in FIGS. 3A-3C, respectively, is taken perpendicular to its respective central axes at an axial distance from the center of its respective femoral head equal to one-half the diameter of its respective femoral head. In FIG. 3A, the cross-section of conventional neck design 40 is generally oval; in FIG. 3B, the cross-section of conventional neck design 50 is generally trapezoidal; and in FIG. 3C, the cross-section of conventional neck design 60 is generally rectangular.

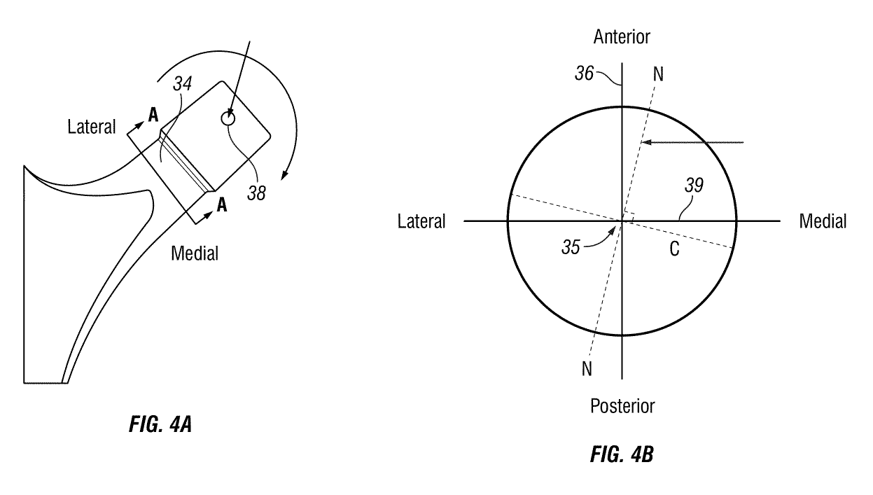

Referring now to FIGS. 4A and 4B, to understand how changes in the geometry of the cross-section of a femoral neck of a femoral implant impacts strength, the stringent ASTM (American Standards of Testing and Measurement) Standard Practice for Cyclic Fatigue Testing of Metallic Stemmed Hip Arthroplasty Femoral Components with Torsion (ASTM F1612-95) will be described with reference to exemplary conventional femoral implant 30. This standard requires that all femoral implants complete, without failing, a minimum number of highly-loaded cycles (5.34 kN) while the implant is positioned according to ISO Standard 7206-6 (rotated in 10° of adduction and 9° of flexion). Under this loading configuration, the neck of the implant (e.g., neck 34) bends about a neutral axis N-N slightly angled relative to the anterior-posterior axis (e.g., axis 36) of the neck. The loading creates off-axis loading of the neck in which the area of greatest stress is near the medial side of an axis C orthogonal to the neutral axis N-N. Although the strength of the neck during this complex bending is generally proportional to its cross-sectional area, not all shapes of the same area will have the same strength. More importantly, strength is proportional to the square of the width of the implant in a direction (along axis 39) orthogonal to the neutral axis N-N. If bending usually occurs about only one axis, it is advantageous to bulk up the cross-section of the structure orthogonal to that axis to enhance strength. In a femoral neck, this would mean lengthening the width along axis C in FIG. 4B. However, the medial side of axis C and the area on the anterior side of axis C are coincidentally in the same region where impingement is likely to occur during straight extension and most flexion maneuvers of the femoral implant. Therefore, an optimization or balance of strength and maneuverability is preferred to create a well-designed femoral neck. Without being limited by this or any particular theory, this optimization involves complex strength and range of motion analysis and can not be achieved by just varying the widths of simple shapes.

Due to the complexity of the hip maneuvers and the off-axis loading of the hip joint described above, there are inherent problems with each of the relatively simple conventional femoral neck designs described with reference to FIGS. 2B and 3A-3C. For example, although the rectangular design 60 (FIG. 3C) provides adequate strength against bending of the neck about the anterior-posterior axis, the medial corners of the neck are common points of impingement for flexion activities and straight extension. Further, the trapezoidal design 50 (FIG. 3B) provides increased range of motion compared to a rectangular cross-section of the same width, but at a loss of considerable strength. Moreover, the oval design 40 (FIG. 3A) increases strength over a circular neck of the same area, but at a loss of range of motion, not unlike the rectangular cross-section.

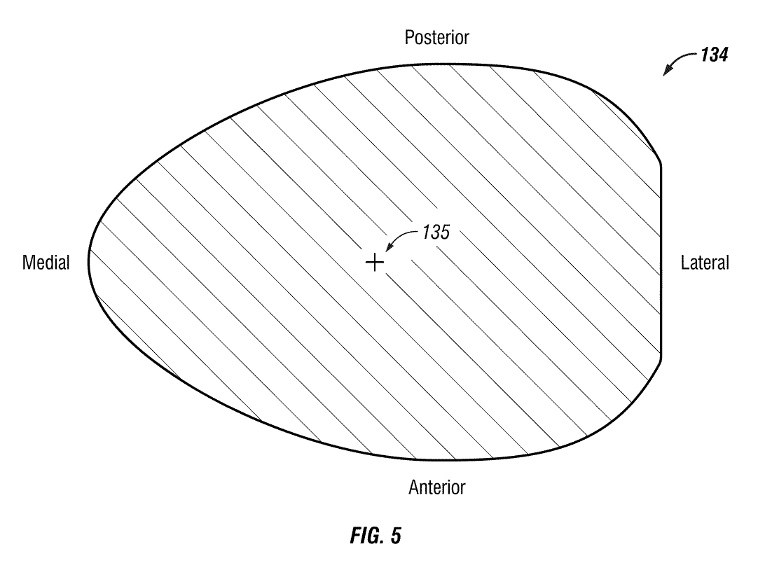

Referring now to FIG. 5, a more recent hip prosthesis design described in U.S. Pat. No. 7,060,102, which is hereby incorporated herein by reference in its entirety, includes a neck 134 having a more ovoid cross-section taken perpendicular to the central or longitudinal axis 135 of neck 134. The ovoid geometry in which the anterior-posterior width of the neck has been reduced, and the medial-lateral width has been increased, attempts to reduce prosthetic impingement without compromising the strength of the component. The major drawback of this design is the inherent trade-off between maximizing motion of the hip during activities involving flexion and internal rotation, leading to impingement between the acetabular cup and the anterior-medial aspect of the prosthetic neck, and maximizing motion during activities with combinations of extension and external rotation, leading to prosthetic impingement over the posterior-lateral aspect of the prosthetic neck. Because the femoral necks of most femoral implants are symmetric about the medial-lateral axis for simplicity and ease of manufacture, significantly reducing these two corners to increase range of motion would entail reducing all four corners, severely compromising the strength of the neck. However, in the design of neck 134, minor reductions in only the two described corners (i.e., anterior-medial aspect and posterior-lateral aspect) sought to balance the improvements in the two described types of activities.

The drawback of this method of neck design is that although the range of motion to prosthetic impingement is increased during most activities in these complex motions, in some hip maneuvers, prosthetic impingement does not even occur at the limit of range of motion. In such activities, soft-tissues such as muscles, tendons, and ligaments restrict further motion of the joint prior to impingement, thereby avoiding prosthetic impingement. Consequently, some reductions in cross-section of certain portions of the neck are unwarranted. Rather, other areas of the cross-section and even the total width of the neck could be reduced further to improve the range of motion during activities that are known to lead to prosthetic impingement without sacrificing strength.

As will be described in more detail below, embodiments described herein address each of the deficiencies above with a neck design that optimizes the range of motion of an artificial hip in real patients, not just the range of motion of the components themselves. Embodiments described herein may be used in any application where an improvement in range of motion of a total hip replacement is desired. In general, the femoral implant includes a symmetric neck portion that is optimized to improve range of motion during the complex maneuvers of the hip. The cross-section of the neck taken perpendicular to the neck axis has a shape with reduced cross-sectional area at portions of the neck known to prosthetically impinge during flexion/internal rotation maneuvers, while maintaining strength by enlarging in area the portions of the neck that do not prosthetically impinge due to soft-tissue restrictions. Most conventional femoral neck designs have either focused on improving the range of motion of simple motions or improving the range of motion of only the prosthetic components themselves. It has been recognized that due to soft-tissue restrictions, the large head to neck ratio (femoral head diameter to neck diameter) and larger neck shaft angle (angle between the center axis of the neck and the long axis of the femur) of an artificial hip, prosthetic impingement on the posterior/lateral side of the neck is considerably less common than on its anterior/medial portion in the human hip. Simply reducing the medial area with an increase in lateral area however, does not always provide the best solution, as previous trapezoidal designs have demonstrated.

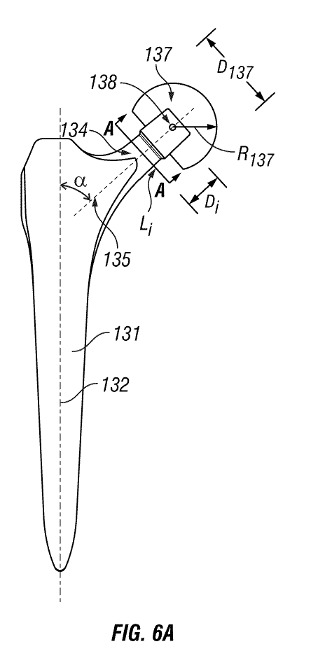

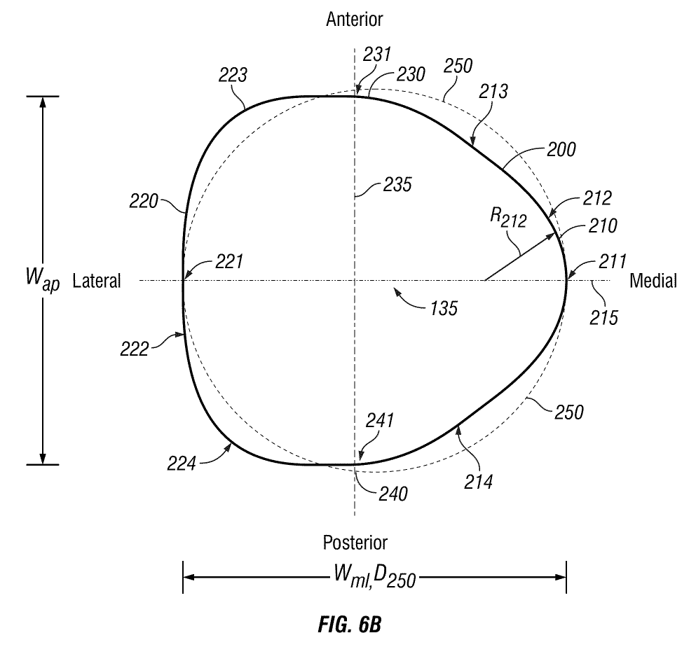

Referring now to FIGS. 6A and 6B, an embodiment of a femoral implant 130 in accordance with the principles described herein is shown. Femoral implant 130 is designed for use with any suitable acetabular cup or socket implant, such as conventional socket implant 20 previously described, to form an artificial hip joint.

As best shown in FIG. 6A, femoral implant 130 includes an elongate femoral stem 131, a femoral neck 134 integral with and extending from the upper end of the femoral stem 131, and a spherical femoral head 137 fixed to the upper end of the femoral neck 134. Femoral stem 131 has a central or longitudinal axis 132 and femoral neck 134 has a central or longitudinal axis 135 that is disposed at an acute angle α relative to axis 132 in front or anterior-posterior view. In addition, spherical femoral head 137 has a geometric center 138 that is equidistant from each point on the spherical surface of the head 137. In addition, spherical femoral head 137 has a diameter D₁₃₇ and a radius R₁₃₇ equal to one-half the diameter D₁₃₇.

To form the prosthetic hip joint, the femoral stem 131 is disposed in the upper end of the femur of a patient with neck 134 and head 137 extending therefrom. Further, the socket implant or acetabular cup (not shown) having a spherical receptacle is disposed in the acetabulum (e.g., acetabulum 14 of pelvis 12). Femoral head 137 is then positioned within the spherical receptacle of the socket implant to form a ball-and-socket artificial hip joint.

As previously described, prosthetic impingement normally occurs at a level L disposed at an axial distance Dᵢ measured parallel to the femoral neck axis (e.g., axis 135) from the center of the femoral head (e.g., center 138 of femoral head 137) that is approximately one-half the diameter of the spherical head (e.g., one-half of diameter D₁₃₇).

Referring now to FIG. 6B, a transverse cross-section 200 of neck 134 is shown. In particular, transverse cross-section 200 is a cross-section of neck 134 perpendicular to axis 135 at level Lᵢ. The outer perimeter of transverse cross-section 200 includes a medial edge 210 (right side in FIG. 6B), a lateral edge 220 opposite medial edge 210 (left side in FIG. 6B), an anterior edge 230 (upper side in FIG. 6B), and a posterior edge 240 opposite anterior edge 230 (lower side in FIG. 6B). Medial edge 210 includes a medial-most point 211, lateral edge 220 includes a lateral-most point 221, anterior edge 230 includes an anterior-most point 231, and posterior edge 240 includes a posterior-most point 241. A medial-lateral axis 215 bisects cross-section 200, is perpendicular to and intersects neck axis 135, and intersects medial-most point 211 along medial edge 210 and lateral-most point 221 along lateral edge 220. Further, transverse cross-section 200 has a maximum medial-lateral width Wₘₗ measured along medial-lateral axis 215 between edges 210, 220. Medial-lateral axis 215 has a mid-point 216 at one-half medial-lateral width Wₘₗ from either edge 210, 220.

Transverse cross-section 200 is symmetric about a medial-lateral axis 215. From the standpoint of increased versatility, the anterior and posterior halves on either side of medial-lateral axis 215 are preferably symmetrical to enable a single femoral implant (e.g., femoral implant 130) to be implanted interchangeably in either the right or left hip joint, thereby reducing the necessity of manufacturing and storing different implants for right and left hip joints.

Referring still to FIG. 6B, an anterior-posterior axis 235 is orthogonal to medial-lateral axis 215 and neck axis 135 and extends from anterior-most point 231 along anterior edge 230 to posterior-most point 241 along posterior edge 240. Transverse cross-section 200 has a maximum anterior-posterior width Wₐₚ measured along anterior-posterior axis 235 between edges 230, 240. In general, medial-lateral width Wₘₗ and anterior-posterior width Wₐₚ of a transverse cross-section of neck 134 may vary depending on the size of femoral implant 130, the material used, and the level along neck axis 135 of the transverse cross-section, but generally range from about 9 mm to about 16 mm. In an exemplary embodiment medial-lateral width Wₘₗ is 12.5 mm and anterior-posterior width Wₐₚ is 12.0 mm. In the embodiments described herein, the ratio of the maximum medial-lateral width Wₘₗ to the maximum anterior-posterior width Wₐₚ is preferably at least 0.9.

Medial edge 210 is curved and comprises three concave arcs—a medial arc 212 that intersects medial-most point 211 and axis 215, a medial-anterior arc 213 that extends from medial arc 212 to anterior-posterior axis 235 at anterior edge 230, and a medial-posterior arc 214 that extends from medial arc 212 to anterior-posterior axis 235 at posterior edge 240. Medial arc 212 of medial edge 210 preferably has a radius or curvature R₂₁₂ greater than or equal to 33% of the maximum anterior-posterior width Wₐₚ. Lateral edge 220 comprises a lateral arc 222 that intersects axis 215, a lateral-anterior arc 223 that extends from lateral arc 222 to anterior-posterior axis 235 at anterior edge 230, and a lateral-posterior arc 224 that extends from lateral arc 222 to anterior-posterior axis 235 at posterior edge 240. Lateral arc 222 is preferably straight or has a relatively large radius of curvature compared to lateral-anterior arc 223 and lateral-posterior arc 224, each of which has a relatively small radius of curvature compared to lateral arc 222. Consequently, the lateral side of transverse cross-section 200 (i.e., the portion of transverse cross-section 200 on the lateral side of anterior-posterior axis 235) is larger in area and is generally more rectangular in shape than the medial side of transverse cross-section 200 (i.e., the portion of transverse cross-section 200 on the medial side of anterior-posterior axis 235). Specifically, the ratio of the total area of transverse cross-section 200 lateral of anterior-posterior axis 235 (to the left of anterior-posterior axis 235 in FIG. 6B) to the total area of transverse cross-section 200 medial of anterior-posterior axis 235 (to the right of anterior-posterior axis 235 in FIG. 6B) is preferably at least 1.2, and more preferably at least 1.4.

Referring still to FIG. 6B, for purposes of comparison and to further define the geometry of transverse cross-section 200, a reference circle 250 is superimposed on transverse cross-section 200. Reference circle 250 has a diameter D₂₅₀ equal to maximum medial-lateral width Wₘₗ, is centered about the intersection of axes 215, 235, and passes through medial-most point 211 of medial edge 210 and lateral edge 220 at medial-lateral axis 215. Thus, as used herein, the phrase “reference circle” refers to a circle having a diameter equal to the maximum medial-lateral width of a femoral neck transverse cross-section and that passes through the lateral-most point of the transverse cross-section and the medial-most point of the transverse cross-section. The geometry and cross-sectional area of transverse cross-section 200 deviates from reference circle 250 by significantly enlarging the lateral corners of transverse cross-section 200 defined by arcs 223, 224, and reducing the medial corners of transverse cross-section 200 defined by arcs 213, 214. Although the medial corners are withdrawn as compared to reference circle 250, as previously described, medial arc 212 preferably has a radius of at least 33% of the maximum anterior-posterior width Wₐₚ. As previously described, reference circle 250 has a diameter D₂₅₀ equal to maximum medial-lateral width Wₘₗ, and thus, reference circle 250 has an area as follows:

$${A}_{reference circle 250} = \frac{{π ( {D}_{250} )}^{2}}{4} = \frac{{π ( {W}_{m l} )}^{2 }}{4}$$

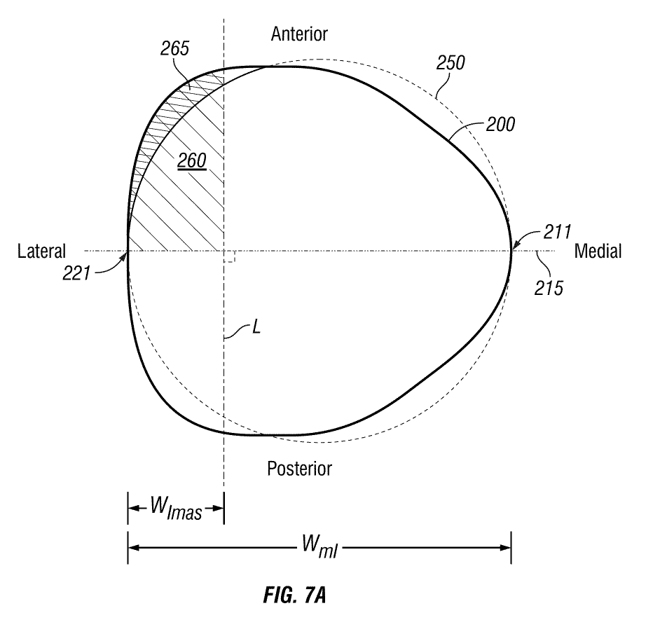

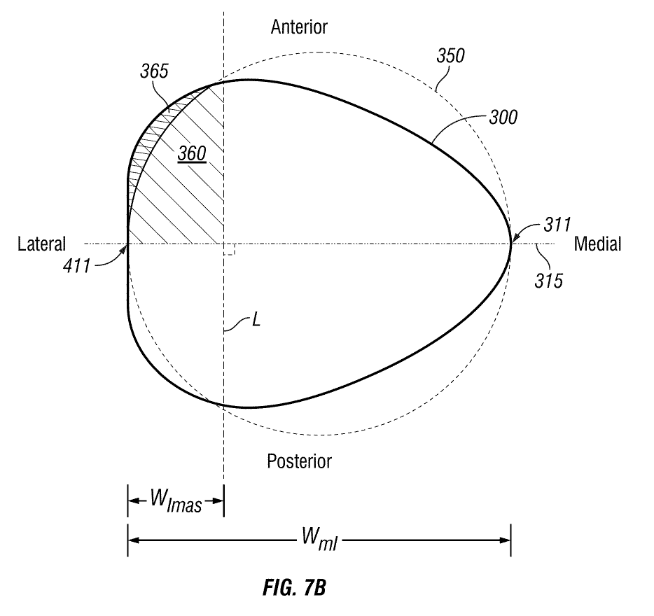

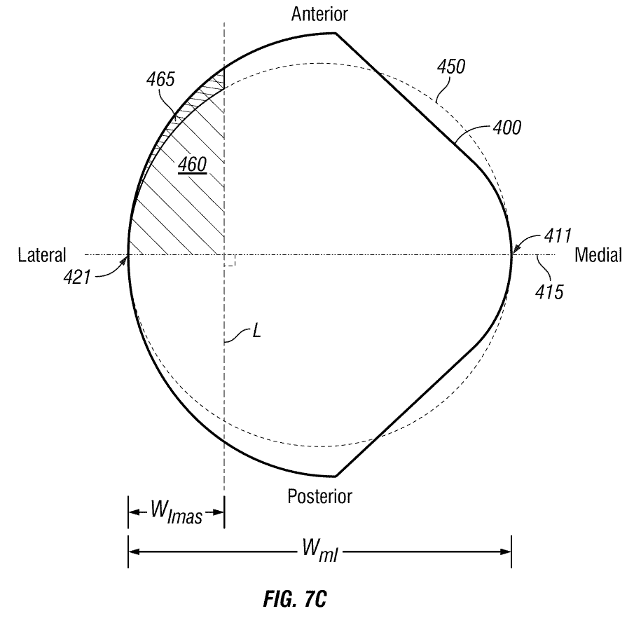

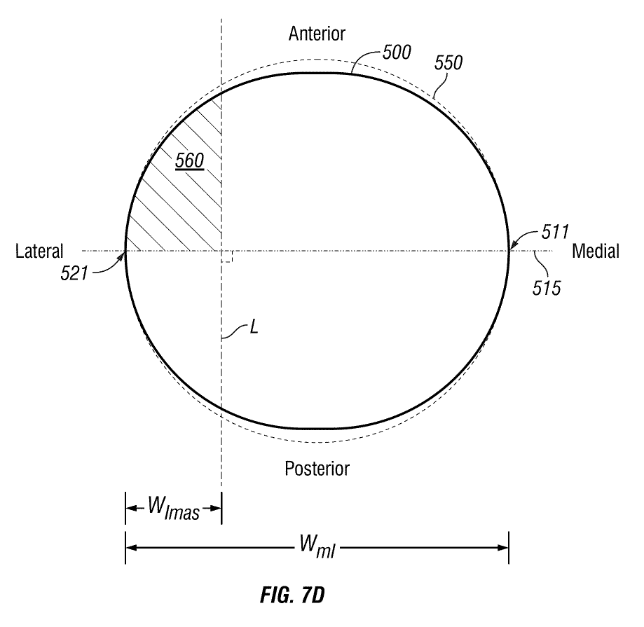

Referring now to FIGS. 7A-7D, transverse cross-section 200 of femoral neck 134 previously described and the transverse cross-sections of three conventional femoral necks are shown. In particular, in FIG. 7A, transverse cross-section 200 of femoral neck 134 is shown; in FIG. 7B a transverse cross-section 300 of the femoral neck disclosed in U.S. Pat. No. 7,060,102 is shown; in FIG. 7C a transverse cross-section 400 of a conventional femoral neck is shown; and in FIG. 7D a transverse cross-section 500 of a conventional oval femoral neck is shown. For purposes of comparison, each transverse cross-section 200, 300, 400, 500 shown in FIGS. 7A-7D was taken at a level Lᵢ disposed at an axial distance Dᵢ measured parallel to the respective central axis of the femoral neck from the center of the respective spherical head fixed to the femoral neck, wherein axial distance Dᵢ is one-half the diameter of the respective spherical head.

Each transverse cross-section 200, 300, 400, 500 has a medial-lateral axis 215, 315, 415, 515, respectively, that bisects cross-section 200, 300, 400, 500, respectively, and passes through a medial-most point 211, 311, 411, 511, respectively, and a lateral-most point 221, 321, 421, 521, respectively. Each transverse cross-section 200, 300, 400, 500 has a maximum medial-lateral width Wₘₗ measured along axis 215, 315, 415, 515, respectively, between medial-most point 211, 311, 411, 511, respectively, and a lateral-most point 221, 321, 421, 521, respectively. For purposes of comparison, a reference circle 250, 350, 450, 550 is superimposed on each transverse cross-section 200, 300, 400, 500, respectively. Each reference circle 250, 350, 450, 550 has a diameter equal to the maximum medial-lateral width Wₘₗ of its respective transverse cross-section 200, 300, 400, 500, and passes through medial-most point 211, 311, 411, 511, respectively, and a lateral-most point 221, 321, 421, 521, respectively.

Each transverse cross-section 200, 300, 400, 500 has a lateral-most anterior segment 260, 360, 460, 560, respectively, with a width Wₗₘₐₛ equal to one-fourth width Wₘₗ shown below.

$${W}_{lmas} = \frac{{W}_{m l}}{4}$$

Lateral-most anterior segment 260, 360, 460, 560 extends along medial-lateral axis 215, 315, 415, 515, respectively, from lateral-most point 221, 321, 421, 521, respectively, to a reference line L perpendicular to medial-lateral axis 215, 315, 415, 515, respectively, and disposed at width Wₗₘₐₛ measured along medial-lateral axis 215, 315, 415, 515, respectively, from lateral-most point 221, 321, 421, 521, respectively. In addition, lateral-most anterior segment 260, 360, 460, 560 extends anteriorly from medial-lateral axis 215, 315, 415, 515, respectively, to the outer perimeter of transverse cross-section 200, 300, 400, 500, respectively. Thus, as used herein, the phrase “lateral-most anterior segment” refers to the lateral-most segment of the anterior half of a femoral neck transverse cross-section extending from the lateral edge to a width that is one-fourth the maximum medial-lateral width of the transverse cross-section.

Referring still to FIGS. 7A-7D, each lateral-most anterior segment 260, 360, 460 includes a laterally expanded portion 265, 365, 465 extending outside reference circle 250, 350, 450, respectively. Thus, as used herein, the phrase “laterally expanded portion” refers to the portion of the lateral-most anterior segment of the transverse cross-section that extends outside the reference circle previously defined. However, as shown in FIG. 7D, lateral-most anterior segment 560 of transverse cross-section 500 does not extend beyond reference circle 550, respectively, and thus, transverse cross-section 500 and lateral-most anterior segment 560 does not include a laterally expanded portion.

The enlarged lateral corners of embodiments described herein may be quantified by comparing the area of the laterally expanded area of embodiments described herein to the area of the laterally expanded areas of the conventional transverse cross-sections. In embodiments described herein (e.g., transverse cross-section 200), the area of the laterally expanded area (e.g., laterally-expanded area 265) is preferably at least 6.5% of the area of one quadrant of the reference circle (e.g., reference circle 250), where the area of one quadrant of the reference circle is one-fourth (¼) the total area of the reference circle, and more preferably greater than 10% of the area of one quadrant of the reference circle. In the embodiment shown in FIG. 7A, the area of laterally expanded area 265 is 10.1 % of the area of one quadrant of reference circle 250. In other words, the ratio of the area of laterally expanded area 265 to one-fourth the area of reference circle 250 is 0.101. However, in conventional transverse cross-section 300, 400, the area of laterally expanded area 365, 465, respectively, is about 6% and 4%, respectively, of the area of one quadrant of reference circle 350, 450. As previously described, conventional transverse cross-section 500 does not have a laterally expanded area.

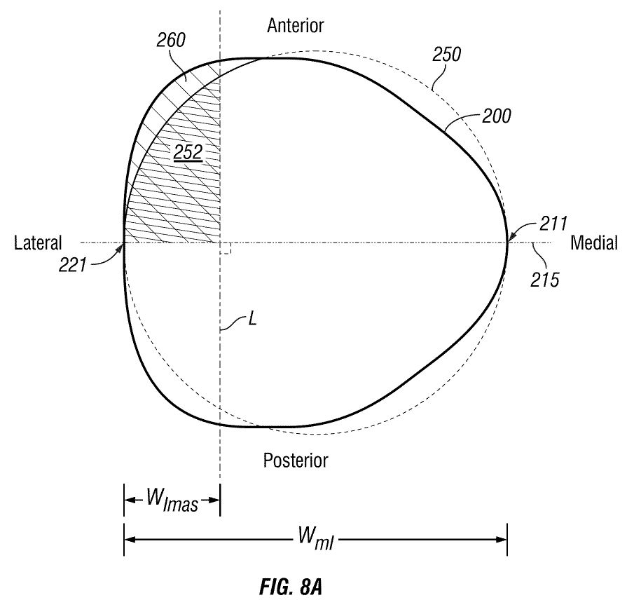

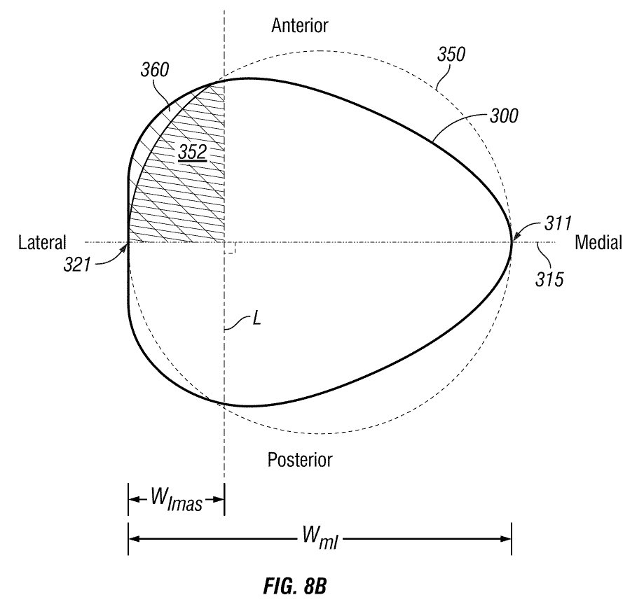

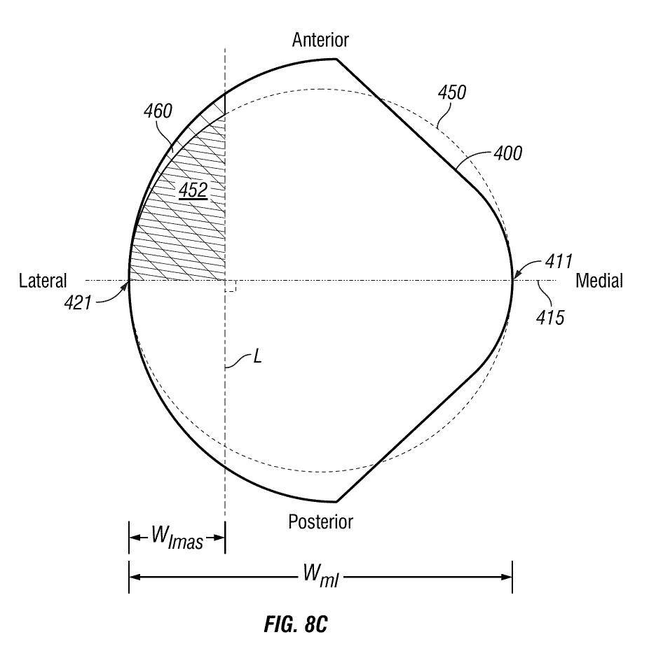

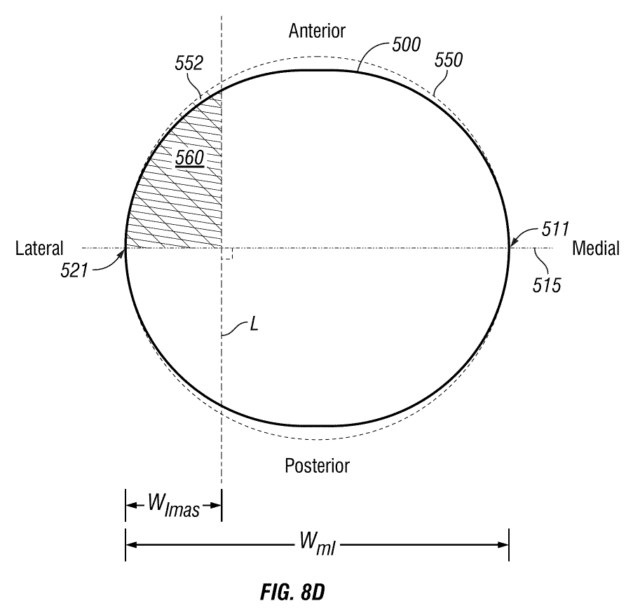

Referring now to FIGS. 8A-8D, the enlarged lateral corners of embodiments described herein may also be quantified by comparing the area of the lateral-most anterior segment to the area of the anterior portion of the reference circle extending between the lateral-most point and reference line L, as referred to herein as the “lateral-most half quadrant of the reference circle.” Thus, as used herein, the phrase “lateral-most half quadrant of the reference circle” refers to the lateral-most segment of the anterior half of the reference circle extending from the lateral-most point to a reference line perpendicular to the medial-lateral axis and disposed at a distance equal to ¼ the diameter of the reference circle. In FIGS. 8A-8D, each reference circle 250, 350, 450, 500 has a lateral-most half quadrant 252, 352, 452, 552, respectively.

In embodiments described herein (e.g., transverse cross-section 200), the area of the lateral-most anterior segment (e.g., lateral-most anterior segment 260) is preferably at least 116% of the area of the lateral-most half quadrant of the reference circle (e.g., the area of lateral-most half quadrant 252 of reference circle 250), and more preferably at least 120% of the area of the lateral-most half quadrant of the reference circle. In the embodiment of transverse cross-section 200 shown in FIG. 8A, the area of lateral-most anterior segment 260 is about 123% of the area of the lateral-most half quadrant 252 of reference circle 250. To the contrary, in FIG. 8B, the area of lateral-most anterior segment 360 of transverse cross-section 300 is 115% of the area of lateral-most half quadrant 352 of reference circle 350; in FIG. 8C, the area of lateral-most anterior segment 460 of transverse cross-section 400 is 110% of the area of lateral-most half quadrant 452 of reference circle 450; and in FIG. 8D, the area of lateral-most anterior segment 560 of transverse cross-section 500 is 96% of the area of lateral-most half quadrant 552 of reference circle 550.

Although the transverse cross-sections shown and described with reference to FIGS. 6A to 8D were taken perpendicular to the femoral neck axis at an axial distance equal to one-half the diameter of the spherical femoral head from the center of the femoral head, the general geometries and configurations, the geometry and configuration of other transverse cross-sections along at other points along a given femoral neck are generally the same between an axial distance of about 12 mm and 22 mm below, or distal to, the center of the femoral head.

While preferred embodiments have been shown and described, modifications thereof can be made by one skilled in the art without departing from the scope or teachings herein. The embodiments described herein are exemplary only and are not limiting. Many variations and modifications of the system and apparatus are possible and are within the scope of the invention. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims.

### Examples

The following examples are given as particular aspects of the embodiments described herein and to demonstrate the practice and advantages thereof. It is understood that the examples are given by way of illustration and are not intended to limit the specification of the claims to follow in any manner.

### Example 1

### Optimization of Femoral Implant Design

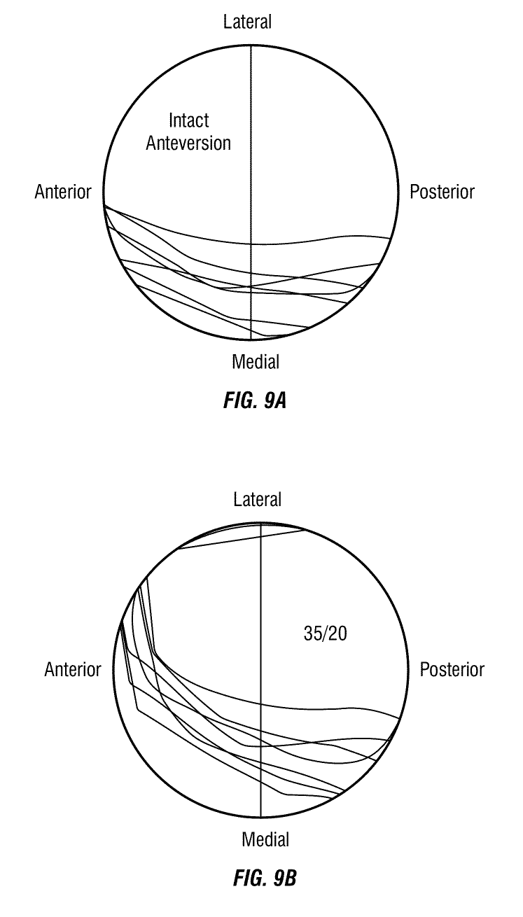

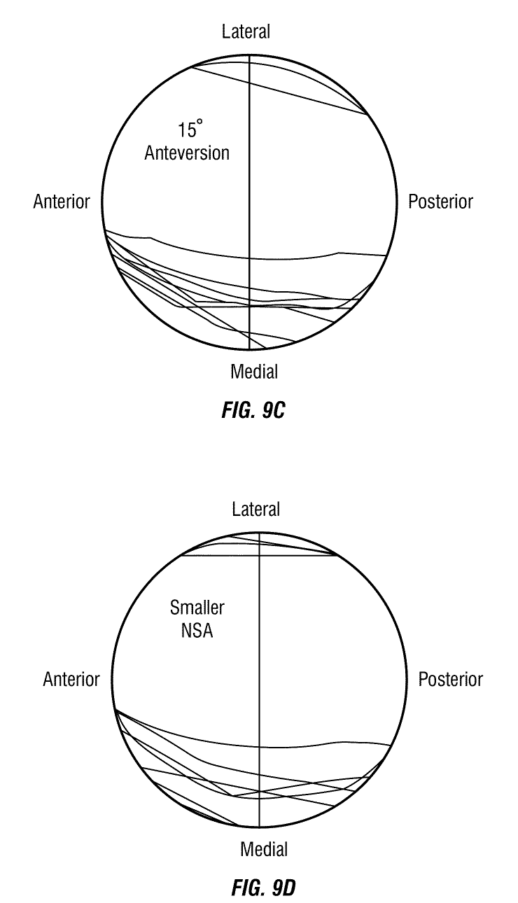

Embodiments described herein were partly derived from experimental data recorded from eight cadaveric hips. The limit of range of motion of each cadaver hip was recorded for twenty-five maneuvers. Then, a typical acetabular cup (32 mm liner) and conventional oversized femoral neck (16 mm diameter) were virtually implanted into each hip and rotated through the same twenty-five maneuvers, allowing the femoral neck to engage and/or penetrate the cup if necessary. The intersecting volume of the femoral neck and cup was then subtracted from the neck for each maneuver, resulting in an “idealized” neck for each specimen that was incapable of prosthetic impingement. The transverse cross-section of all eight “idealized” necks were then superimposed and averaged. This procedure was performed for 4 different sets of component orientations as follows:
1. The acetabular cup placed in 35° of inclination and 20° of anteversion with the stem anteverted the same amount as the intact femoral neck.
2. The acetabular cup placed in 45° of inclination and 20° of anteversion with the stem anteverted the same amount as the intact femoral neck.
3. The acetabular cup placed in 45° of inclination and 20° of anteversion with the stem in 15° of anteversion.
4. The acetabular cup placed in 35° of inclination and 20° of anteversion with the stem anteverted the same amount as the intact femoral neck, but abducted 4° (the equivalent of reducing the neck-shaft angle (NSA) of the femur).
   The resulting cross-sections for each of the four component orientations 1, 2, 3, and 4 above are shown in FIGS. 9A-9D, respectively. The majority of the subtracted/impinging area occurred in the anterior/medial corner of the neck. There were very few cases where the lateral corners impinged with the acetabular cup.

The lack of posterior/lateral impingement was further supported by reviewing the rotational data of the eight hips. The average external rotation in extension of all hips was 25.3±3.7°. This maneuver was very similar to both the pivot and roll maneuvers. The tests showed that a typical 12 mm diameter femoral stem with a 32 mm head, anatomically positioned in the femur and articulating with a cup at 45° of inclination and 20° of anteversion, was capable of over 60° of external rotation in extension, and over 40° of external rotation during pivoting and rolling.

### Example 1

### Range of Motion Comparison

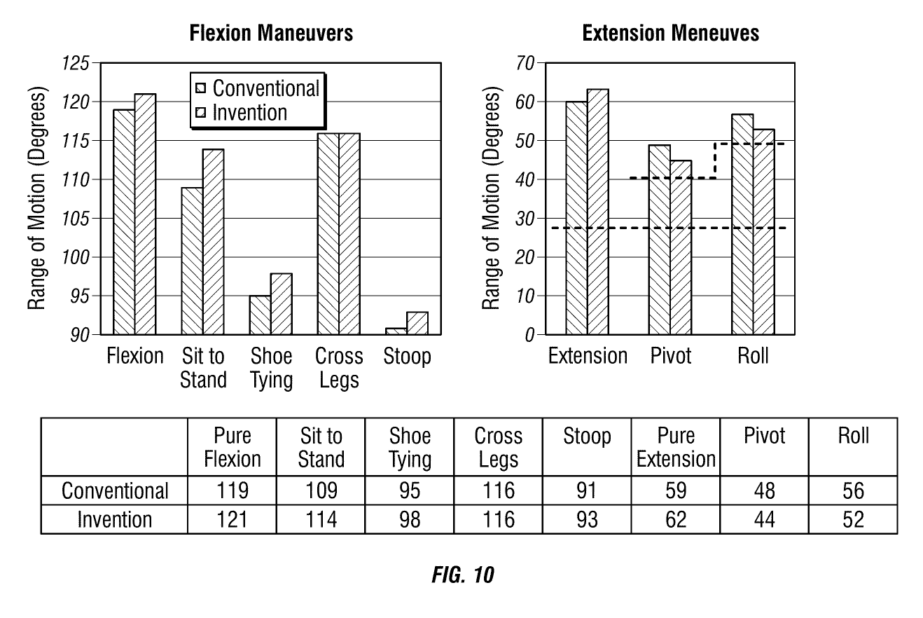

The range of motion of embodiments of femoral necks designed in accordance with the principles described herein were also compared to a conventional 12 mm conical neck of similar strength. As shown in FIG. 10, the increase in range of motion of maneuvers highly susceptible to dislocation such as sit to stand and shoe-tying was about 5° and about 3°, respectively.

FIG. 10 also illustrates an apparent decrease in external rotation/extension maneuvers to prosthetic impingement during pivoting and rolling maneuvers of embodiments described herein compared to the conventional 12 mm neck. This difference is generally irrelevant, however, as both necks easily surpassed the limits of each maneuver as estimated by the experimental data. For example, normal patients considerably younger (and likely more flexible) than the typical total hip patient (49.7±5.0 yrs. vs. 65-70 yrs.) have limits of external rotation during rolling and pivoting still below those of the embodiments described herein and the conventional 12 mm neck.

### Example 2

### Strength Analysis of Femoral Implant Design

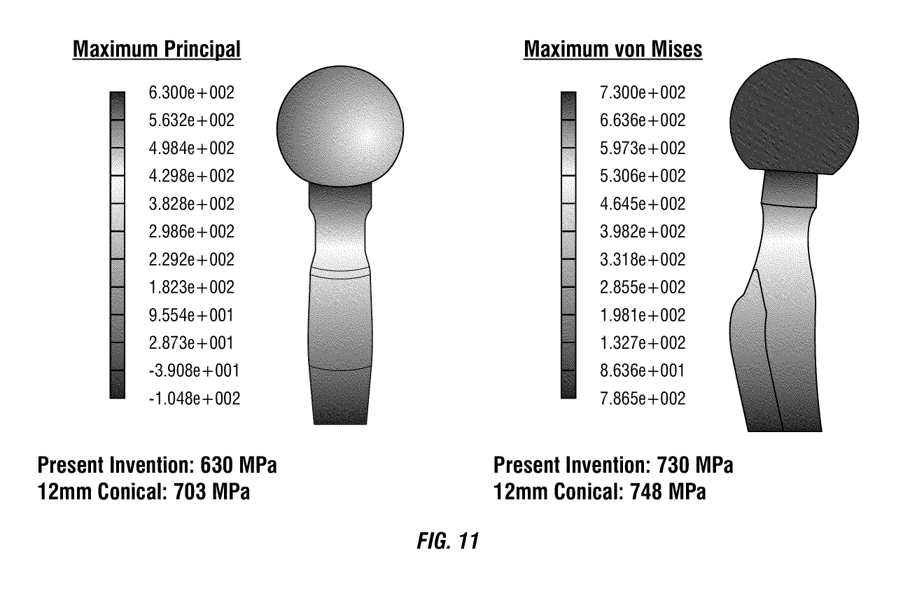

Computer modeling and testing of a femoral neck constructed in accordance with the principles described herein was performed to ensure sufficient strength to pass the stringent ASTM standard F1612-95 described previously. A 3D computer model of the neck was placed on a standard stem model. The maximum stresses in the neck were calculated using finite element analyses, and were compared virtually with a conventional 12 mm conical neck known to have sufficient strength. Each model was meshed in 3D using tetrahedral elements (average size=1.0 mm). Each model was then positioned in 10° of adduction and 9° of flexion and constrained below the stem's osteotomy as required in ISO Standard 7206-6. A 5340N load was applied inferiorly to the center of the head using the worst case scenario for head offset (head position along the neck axis). As shown in FIG. 11, the maximum principal stresses and the maximum von Mises stresses were compared for both necks. Regardless of the failure criteria, lower maximum stresses were observed in the neck designed in accordance with the principles described herein as compared to the conventional 12 mm conical neck (maximum principal: 630 vs. 703 MPa, maximum von Mises: 730 vs. 748 MPa).

What is claimed is:

1. A prosthetic femoral implant for use in hip arthroplasty, the femoral implant comprising:
an elongate femoral stem;
a femoral neck having a central axis, a first end integral with the femoral stem, and a second end distal the femoral stem;
a spherical femoral head coupled to the second end of the femoral neck;
wherein a transverse cross-section of the femoral neck taken perpendicular to the central axis has an outer perimeter including a medial edge, a lateral edge opposite the medial edge, an anterior edge, and a posterior edge opposite the anterior edge;
wherein the transverse cross-section of the femoral neck includes:
a medial-lateral axis bisecting the transverse cross-section into an anterior half and a posterior half, wherein the medial-lateral axis intersects a medial-most point along the medial edge and a lateral-most point along the lateral edge, and wherein the transverse cross-section has a maximum medial-lateral width Wₘₗ measured along the medial-lateral axis between the medial edge and the posterior edge;
an anterior-posterior axis perpendicular to the medial-lateral axis and extending from a posterior-most point along the posterior edge to an anterior-most point along the anterior edge, wherein the transverse cross-section has a maximum anterior-posterior width Wₐₚ measured along the anterior-posterior axis between the posterior edge and the anterior edge;
wherein the anterior half of the transverse cross-section includes a lateral-most anterior segment extending from the lateral-most point to a reference line, wherein the reference line is perpendicular to the medial-lateral axis and crosses the medial-lateral axis at a distance Dₗ measured along the medial-lateral axis from the lateral-most point, wherein the distance Dₗ is equal to one-fourth the maximum medial-lateral width Wₘₗ;
wherein a reference circle bisected by the medial-lateral axis and passing through the medial-most point and the lateral-most point has a diameter equal to the maximum medial-lateral width Wₘₗ of the transverse cross-section and an area A₁;
wherein the lateral-most anterior segment of the transverse cross-section includes a laterally expanded area extending outside the reference circle, the laterally expanded area having an area A₂;
wherein the area A₂ of the laterally expanded area is at least 7% of one-fourth of the area A₁ of the reference circle.

2. The femoral implant of claim 1, wherein the area A₂ of the laterally expanded area is at least 10% of one-fourth of the area A₁ of the reference circle.

3. The femoral implant of claim 1, wherein the ratio of the area of the transverse cross-section lateral the anterior-posterior axis to the area of the transverse cross-section medial the anterior-posterior axis is greater than 1.2.

4. The femoral implant of claim 3, wherein the ratio of the area of the transverse cross-section lateral the anterior-posterior axis to the area of the transverse cross-section medial the anterior-posterior axis is greater than 1.4.

5. The femoral implant of claim 4, wherein the medial edge includes a medial arc that passes through the medial-most point, wherein the medial arc has a radius of curvature that is at least 33% of the maximum anterior-posterior width Wₐₚ.

6. The femoral implant of claim 4, wherein the lateral edge includes a lateral arc that passes through the lateral-most point, wherein the lateral arc has a radius of curvature that is greater than the radius of curvature of the medial arc.

7. The femoral implant of claim 1, wherein the ratio of the maximum medial-lateral width Wₘₗ to the maximum anterior-posterior width Wₐₚ is at least 0.9.

8. A prosthetic femoral implant for use in hip arthroplasty, the implant comprising:
an elongate femoral stem;
a femoral neck having a central axis, a first end integral with the femoral stem, and a second end distal the femoral stem;
a spherical femoral head coupled to the second end of the femoral neck;
wherein a transverse cross-section of the femoral neck taken perpendicular to the central axis has an outer perimeter including a medial edge, a lateral edge opposite the medial edge, an anterior edge, and a posterior edge opposite the anterior edge;
wherein the transverse cross-section of the femoral neck includes:
a medial-lateral axis bisecting the transverse cross-section into an anterior half and a posterior half, wherein the medial-lateral axis intersects a medial-most point along the medial edge and a lateral-most point along the lateral edge, and wherein the transverse cross-section has a maximum medial-lateral width Wₘₗ measured along the medial-lateral axis between the medial edge and the posterior edge;
an anterior-posterior axis perpendicular to the medial-lateral axis and extending from a posterior-most point along the posterior edge to an anterior-most point along the anterior edge, wherein the transverse cross-section has a maximum anterior-posterior width Wₐₚ measured along the anterior-posterior axis between the posterior edge and the anterior edge;
wherein the anterior half of the transverse cross-section includes a lateral-most anterior segment extending from the lateral-most point to a reference line, wherein the reference line is perpendicular to the medial-lateral axis and crosses the medial-lateral axis at a distance Dₗ measured along the medial-lateral axis from the lateral-most point, wherein the distance Dₗ is equal to one-fourth the maximum medial-lateral width Wₘₗ;
wherein the lateral-most anterior segment of the transverse cross-section has an area A₁;
wherein a reference circle bisected by the medial-lateral axis and passing through the medial-most point and the lateral-most point has a diameter equal to the maximum medial-lateral width Wₘₗ of the transverse cross-section;
wherein the reference circle includes a lateral-most half quadrant extending from the lateral-most point along the lateral edge to the reference line, the lateral-most half quadrant of the reference circle having an area A₂;
wherein the area A₁ of the lateral-most anterior segment of the transverse cross-section is at least 116% of the area A₂ of the lateral-most half quadrant of the reference circle.

9. The femoral implant of claim 8, wherein the area of the lateral-most anterior segment of the transverse cross-section is at least 120% the area of the lateral-most half quadrant of the reference circle.

10. The femoral implant of claim 8, wherein the ratio of the area of the transverse cross-section lateral the anterior-posterior axis to the area of the transverse cross-section medial the anterior-posterior axis is greater than 1.2.

11. The femoral implant of claim 10, wherein the ratio of the area of the transverse cross-section lateral the anterior-posterior axis to the area of the transverse cross-section medial the anterior-posterior axis is greater than 1.4.

12. The femoral implant of claim 8, wherein the medial edge includes a medial arc that passes through the medial-most point, wherein the medial arc has a radius of curvature that is at least 33% of the maximum anterior-posterior width Wₐₚ.

13. The femoral implant of claim 12, wherein the lateral edge includes a lateral arc that passes through the lateral-most point, wherein the lateral arc has a radius of curvature that is greater than the radius of curvature of the medial arc.

14. The femoral implant of claim 8, wherein the ratio of the maximum medial-lateral width Wₘₗ to the maximum anterior-posterior width Wₐₚ is at least 0.9.
